(19) United States
(12) Patent Application Publication
(10) Pub. No.: US 2010/0316590 A1
(43) Pub. Date: Dec. 16, 2010

(54) COMPOSITIONS AND METHODS RELATED TO POLOXAMER COPOLYMER MEMBRANE SEALANT

(75) Inventor: Rakez Kayed, Galveston, TX (US)

Correspondence Address: FULBRIGHT & JAWORSKI L.L.P., 600 CONGRESS AVE., SUITE 2400, AUSTIN, TX, 78701, US

(73) Assignee: THE BOARD OF REGENTS OF THE UNIVERSITY OF TEXAS SYSTEM, Austin, TX (US)

(21) Appl. No.: 12/814,953

(22) Filed: Jun. 14, 2010

Related U.S. Application Data
(60) Provisional application No. 61/186,599, filed on Jun. 12, 2009.

(51) Int. Cl. A61K 31/765; C12N 5/071; A61P 25/28

(52) U.S. Cl. 424/78.37; 435/325

(57) ABSTRACT

Embodiments of the invention include the treatment of amyloid oligomer toxicity by administering a membrane sealant co-polymer, for example poloxamer 188 (P188).

This application claims priority to U.S. Provisional Patent application Ser. No. 61/186,599 filed Jun. 12, 2009, which is incorporated herein by reference in its entirety.

### DESCRIPTION OF THE INVENTION

I. Field of the Invention

Embodiments of this invention are directed generally to biology and medicine, particularly it is related to treatment of protein folding disorders.

II. Background

Protein mis-folding and accumulation has been shown to be a critical feature of a variety of different amyloid-related degenerative diseases, including Alzheimer's disease (AD), Parkinson's disease (PD), Huntington's disease (HD) and prion diseases. Regardless of protein sequence and disease, increasing evidence has implicated amyloid oligomers as the primary toxic species rather than the fibrillar end-products that accumulate^{1; 2; 3; 4; 5}. The reason that amyloid oligomers are generically toxic may be due to the fact that they share a common structure, suggesting that they also share a common mechanism(s) of toxicity. Indeed, even oligomers formed by proteins that are not disease-related have proven to be equally toxic as disease-related oligomers¹. If a common mechanism of pathogenesis is set in motion by these oligomers it would follow that they would act on the same primary target. In order to be considered a potential target, it would have to be accessible to all different types of amyloid oligomers, those residing intracellularly as well as extracellularly. Therefore, the most obvious target is the plasma membrane, the interface between the two compartments.

A growing body of evidence points to membrane permeabilization by amyloid oligomers as a common mechanism of pathogenesis in amyloid-related degenerative diseases^{2; 3; 4; 5; 6, 7; 8; 9}. An increase in membrane permeability and intracellular calcium concentration has long been associated with amyloid pathogenesis, though questions remain as to the mechanism underlying these observations^{10; 11}. Numerous amyloidogenic proteins and peptides including Aβ, α-synuclein, IAPP, and polyglutamine have been reported to form discrete pores or ion-specific channels in membranes in their prefibrillar conformations^{7; 12; 13, 14; 15; 16; 17}. These data culminated in what came to be known as the “channel hypothesis,” implicating amyloid peptide channels in the pathogenic ion dysregulation observed in degenerative disease¹⁸. Other studies using homogeneous preparations of amyloid oligomers have also reported membrane permeabilization, but do not observe the types of channels previously described^{6; 8; 19; 20; 21; 22}. These studies suggest that membrane permeabilization caused by amyloid oligomers is due to defects in the lipid bilayer, rather than the formation of discrete proteinaceous pores.

A number of other pathological conditions are also associated with lipid bilayer defects or damage, including membrane permeabilization in tissues exposed to ionizing radiation²³ or electrical shock^{24; 25}, myocardial tissues that accumulate lysophosphotidylcholine (LPC) during ischemia²⁶, traumatic brain injury²⁷, and mechanical chaffing of the corneal epithelium²⁸.

There remains a need for compositions and methods for treating these protein folding disorders.

### SUMMARY OF THE INVENTION

Amyloid oligomers and protofibrils increase cell membrane permeability, eventually leading to cell death. Amyloid oligomer toxicity and membrane permeabilization can be reversed using the membrane sealant co-polymer, for example poloxamer 188 (P188). The amyloid oligomer induced toxicity is caused by defects in lipid bilayer of the type that are sealed by a poloxamer.

Embodiments include compositions and methods of using polymer-based membrane sealants to prevent or reverse amyloid oligomer toxicity. Because the ability to permeabilize membranes is a generic property of amyloid oligomers, this therapeutic approach can be effective for the treatment of many degenerative diseases caused in part by the interaction of misfolded proteins with cell membranes, as in Alzheimer's disease, type II diabetes, and a host of others.

P188 is tri-block copolymer composed of a hydrophobic domain sandwiched between two hydrophilic domains with the following general structure: H(CH₂CH₂)ₐ(OCHCH₃CH₂)_{b}(OCH₂CH₂)ₐOH²³. In certain aspects “a” can be 50, 60, 70, 80, 90, or 100 including all values and ranges there between, and “b” can be 20, 21, 22, 23, 24, 25, 26, 27, 28, 29, 30, 31, 32, 33, 34, 35, 36, 37, 38, 39, or 40, including ranges there between. P188 is non-toxic, non-ionic synthetic surfactant that belongs to a family of more than 30 different surface active agents. Clinically, poloxamers have been used as drug delivery agents, specifically, as enhancers of blood-brain barrier (BBB) penetration. P188 has been reported to cross the BBB and rescue glutamate toxicity in rats²⁹. Additionally, ¹⁴C-labeled P188 delivered peripherally to rats was detectable in brain 4 h later³⁰. Among its other uses are as an emulsifier for artificial blood and as an anticoagulant in microcirculation³¹. It is also a known surfactant and wetting agent for antibiotics³².

Also contemplated are other polymers that contain variants of this formula but still retain the membrane sealing capability demonstrated, for example, by P188. In certain aspects a polymer of the can have the structure H(Y)a(X)b(Z)a, wherein Y is selected from a branched or unbranched alkyl chain having 1, 2, 3, 4, 5, 6, 7, 8, 9, or 10 carbons; X is selected from a group comprising OCHCH₃(CH₂) having 1, 2, 3, 4, 5, 6, 7, 8, 9, or 10 CH₂ groups; and Z is selected from a group comprising OCH(CH₂) having 1, 2, 3, 4, 5, 6, 7, 8, 9, or 10 CH₂ groups. In certain apsects “a” can be 50, 60, 70, 80, 90, or 100 including all values and ranges there between, and “b” can be 20, 21, 22, 23, 24, 25, 26, 27, 28, 29, 30, 31, 32, 33, 34, 35, 36, 37, 38, 39, or 40, including ranges there between.

P188 has been reported to effectively restore the integrity of damaged membranes, including monolayers, bilayers, and cell membranes, by inserting into them and increasing lipid packing density³³. The resulting sealing effect is modulated by membrane surface pressure: the insertion of P188 into a monolayer increases as surface pressure decreases³³. Moreover, P188 seems to exert a localized effect in that it only adsorbs onto damaged membranes, where lipid packing density is reduced³³. Pretreatment of cell membranes with P188 also served to mechanically reinforce them and prevent injury²⁶.

In certain aspects it is the ability of P188 or similar molecules to repair cell membranes and rescue cell viability following permeabilization by a variety of amyloid oligomers that is exploited for therapeutic purposes. P188 when added to cells following exposure to various kinds of amyloid oligomers promotes cell survival and reduces the bidirectional leakage of molecules across the damaged membrane. Amyloid oligomers cause defects in the lipid bilayer itself that result in increased membrane permeability. P188 serves to temporarily repair these membrane defects and reinforce the cell membrane in the face of peixneabilization caused by oligomers while the cell's own innate repair mechanisms patch the disrupted membrane via exocytic vesicle fusion.

In certain aspects, an effective amount of a membrane-sealing agent is administered to a patient in need of such therapy. In certain aspects the membrane sealing agent is administered intravascularly, intraarterially, intracranially, in the central nervous systems,

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The terms “inhibiting,” “reducing,” or “prevention,” or any variation of these terms, when used in the claims and/or the specification includes any measurable decrease or complete inhibition to achieve a desired result.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.” It is also contemplated that anything listed using the term “or” may also be specifically excluded.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the term “patient” or “subject” refers to a living mammalian organism, such as a human, monkey, cow, sheep, goat, dogs, cat, mouse, rat, guinea pig, or transgenic species thereof. In certain embodiments, the patient or subject is a primate. Non-limiting examples of human subjects are adults, juveniles, infants and fetuses.

“Pharmaceutically acceptable” means that which is useful in preparing a pharmaceutical composition that is generally safe, non toxic and neither biologically nor otherwise undesirable and includes that which is acceptable for veterinary use as well as human pharmaceutical use.

The term “effective,” as that term is used in the specification and/or claims, means adequate to accomplish a desired, expected, or intended result, such treating or ameliorating symptoms or conditions related to a protein mis-folding or protein aggregation disorder.

“Therapeutically effective amount” means that amount which, when administered to an animal for treating a disease, is sufficient to effect such treatment for the disease.

“Treatment” or “treating” includes: (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology, and/or (3) effecting any measurable decrease in a disease in a subject or patient that is experiencing or displaying the pathology or symptomatology of the disease.

As used herein, the term “water soluble” means that the compound dissolves in water at least to the extent of 0.010 mole/liter or is classified as soluble according to literature precedence.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: P188 promotes cell survival of cells treated with Aβ42 oligomers. SH-SY5Y cells treated with Aβ42 oligomers (2.5 μM) then supplemented with 2 μg P188 (final concentration, 20 ng/μl) after 15, 45, or 90 min. Cells continued to incubate for 3.5 h and a final toxicity reading was taken. The longer the cells were exposed to oligomers before P188 addition, the less effective the treatment was at reversing the damage to cells and ultimately rescuing them. When applied at 15 min, P188 was significantly more effective at rescuing cells than after 90 min of oligomer treatment.

FIG. 2: Polymer rescues cells treated with a variety of amyloid oligomers. SH-SY5Y cells were treated with oligomers (2.5 μM) for 15 min before 2 μg P188 (final concentration, 20 ng/μl) was added to them. Cells were then incubated for 1 h before performing MTT assay. P188 restored viability of cells treated with Aβ42, IAPP, α-synuclein, prion, or Aβ40 oligomers.

FIG. 3: (3A); P188 rescues primary neurons from Aβ42 oligomer-induced toxicity. Primary rat hippocampal neurons treated with 2.5 μM Aβ42 oligomers showed increased survival when supplemented with P188, as measured by MTT. Addition of 2 μg P188 to the media (final concentration, 20 ng/μl) 45 min after oligomer application nearly doubled cell survival. (3B); P188 does not protect neurons from glutamate toxicity. No significant improvement in cell survival upon treatment with P188 following glutamate treatment (25 μM, 100 μM, 250 μM), as determined by MTT assay. (3C); P188 rescues the toxicity induced by Aβ42 oligomers independent of the method of preparation. P188 rescued the toxicity induced by Aβ42 oligomers prepared by NaOH-based method; the results were similar to the rescue observed for oligomers prepared by the HFIP-based method (FIG. 1). SH-SY5Y cells treated with Aβ42 oligomers prepared by the NaOH method (2.5 μM) then supplemented with 2 μg P188 (final concentration, 20 ng/μl) after 15, 45, or 90 min, then assayed by MTT.

FIG. 4: Oligomeric conformation is preserved in the presence of P188. Dot blot shows amyloid oligomers (1-5; Aβ42, Aβ40, prion, IAPP, and α-synuclein, respectively) in the presence and absence of P188. (A); Oligomeric conformation was confirmed by detection with anti-oligomer (A11) antibody; P188 was not recognized by this antibody. Oligomers incubated with P188 in the presence of absence of media for either 30 min or 60 min. (B); Graph shows quantification of A11 signal intensity is not significantly different in the presence of P188, and is reduced in the presence of media as a result of dilution only. Results were confirmed by ELISA using A11 antibody as graphed in (C); There was no significant difference in A11 binding to Aβ42 oligomers alone or those incubated with P188. Finally, in (D); Western blot of Aβ42 and IAPP oligomers with (lane 1) and without (lane 2) P188 probed with A11 showed that the oligomeric conformation is maintained in the presence of P188, regardless of sequence.

FIG. 5: Rescue of oligomer-induced membrane-damaged cells by P188 is specific. Toxicity induced by Aβ42 oligomers (2.5 μM) is not alleviated by addition of the non-specific poloxamer (P407), as assayed by AlamarBlue. Cells were pretreated with oligomers for 15 minutes after which 20 ng/μl P407 was added to each well and cell viability was assayed 3 h later.

FIG. 6: Effect of P188 is concentration-dependent. SH-SY5Y cells were treated with 4 μM Aβ42 oligomers for 15 minutes before addition of different concentrations of P188, ranging from 8.5-42 ng/μl. Cell viability assayed by AlamarBlue indicates that increasing concentrations of P188 result in increasing cell survival in the presence of oligomers. P188 significantly improved cell viability at 25.5 ng/μl (p=0.05), 34 ng/μl (p=0.021), and 42 ng/μl (p=0.024), compared to oligomer-alone treated controls.

FIG. 7: P188 reseals leaking cell membranes damaged by exposure to amyloid oligomers. EthD-1 dye leakage into cells treated with 2.5 μM Aβ42 oligomers is blocked following addition of 20 ng/μl P188. After 4 minutes P188 to stops the leakage of the dye as indicated by the stabilization in fluorescence, while those cells treated with only oligomers continued to take up dye, indicative of their damaged membranes (p=0.011, 0.001, 0.016, 0.006, and 0.020 for 6 min, 8 min, 10 min, 12 min and 14 min, respectively).

### DETAILED DESCRIPTION OF THE INVENTION

The maintenance of plasma membrane integrity is critical for cell viability, since it controls the exchange of materials between the cell and its surrounding environment. Therefore, restoration of membrane integrity following an injurious stimulus, such as amyloid oligomer-induced permeabilization, is crucial if the cell is to maintain homeostasis and viability. Although numerous studies have reported the ability of amyloids to increase the conductivity of lipid bilayers and cells, the mechanism for this change is not clear. Early studies reported the formation of specific, discrete channels in membranes that show evidence of ion selectivity and can be inhibited by specific channel blockers^{13; 37}. Additionally, some have reported that amyloid oligomers or protofibrils specifically increase the permeability of lipid bilayers, but do so in a non-selective fashion in the absence of discrete or unitary conductances^{6; 8}. This is consistent with observations that amyloid oligomers or protofibrils cause the leakage of fluorescent dyes from lipid vesicles^{19; 38} and the observation that oligomers induce defects in the lipid bilayer radiating out from the site of contact with oligomers^{20; 39}. Most recently, amyloid oligomers have been shown to share structural homology with pore-forming proteins, suggesting that they may also then share the same mechanism of membrane permeabilization⁴⁰.

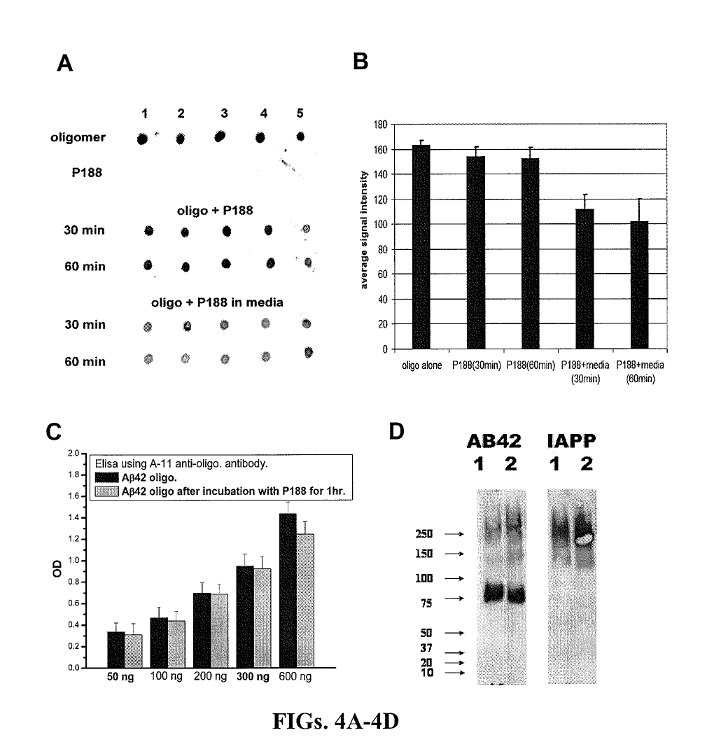

The inventors demonstrate that this disruption in membrane impermeability, leakage of cell contents, and subsequent cell death can be reversed by the application of membrane sealining polymers, e.g., P188, following acute cell injury by amyloid oligomers. The rescue by P188 is specific, and does not depend on its interaction with and disruption of the oligomers themselves (FIG. 4).

P188 has been previously shown to promote the resealing of membranes that contain holes or defects in lipid packing in several different in vivo and in vitro experimental models. P188 has been shown to selectively adsorb to monolayers with decreased lipid surface pressure-area and intercalate in areas of defective in lipid packing. Upon restoration of normal surface pressures, the poloxamer is squeezed out of the monolayer³³. Moreover, irradiated, dye-loaded skeletal muscle cells treated with P188 were almost completely protected from an increase in membrane permeability^{23; 41}. P188 was also successful at restoring cell viability in a dose-dependent manner to PC12 neuronal cells acutely following mechanical membrane damage²⁷. Some reports also demonstrate that pretreatment of membranes may also protect them from injurious electroporation. Others have reported that addition of P188 to synthetic lipid bilayers raised their electroporation threshold and increased membrane stiffness^{24; 42}. Additional evidence in isolated rat hearts demonstrates that membrane permeabilization by lysophosphatidylcholine (LPC) is inhibited by pretreatment with P188, which was time and concentration dependent²⁶. This work indicates that the mechanism of action of poloxamer P188 in resealing damaged membranes is due to its ability to fill lipid packing defects in the bilyer⁴³.

The fact that poloxamer P188 is also capable of resealing membranes permeabilized by amyloid oligomers and preventing cell toxicity is consistent with the interpretation that oligomer-induced membrane damage is due to the creation of defects in lipid packing. However, this does not exclude the possibility that the ability of P188 to inhibit membrane permeabilization may also be due to a previously unrecognized ability of P188 to inhibit channels formed by amyloids, although this is not the simplest interpretation. Still, several recent reports concur that amyloid oligomers do not seem to be forming amyloid channels within membranes, as single channels remain undetectable^{8; 22}. Rather, oligomers seem to increase bilayer conductance by thinning the membrane and lowering the dielectric barrier.

The resealing of cell membranes promotes cell survival in response to membrane damage. Cells have membrane repair mechanisms in place that allow them to recover from transient losses of membrane integrity. These are initiated by the sudden rise in calcium due to influx through the puncture sites²⁸. Plasma membrane repair is mediated by exocytic vesicle fusion triggered by such calcium influx⁴⁴. This attempted rescue may be insufficient, as cells can still enter apoptosis due to exceeding the signaling threshold that is initiated by the initial traumatic event. Growing evidence points to a disruption of intracellular Ca²⁺ homeostasis in AD and other amyloidogenic diseases^{45; 46}, and elevated intracellular Ca²⁺ levels are known to trigger apoptosis and/or excessive phosphorylation of key proteins that ultimately lead to cell death^{10; 47}. Pre-fibrillar amyloid aggregates have been shown to elevate cytosolic Ca²⁺ in neurons^{1; 46}. Extracellular applications of oligomeric amyloid proteins and peptides induce rapid rises (within seconds) in cytosolic free Ca²⁺, whereas equivalent amounts of monomers and fibrils show no detectable change in intracellular Ca²⁺ levels⁶. The Ca²⁺ influx is likely coupled with exocytic vesicle fusion as an attempt to repair the leaky membrane.

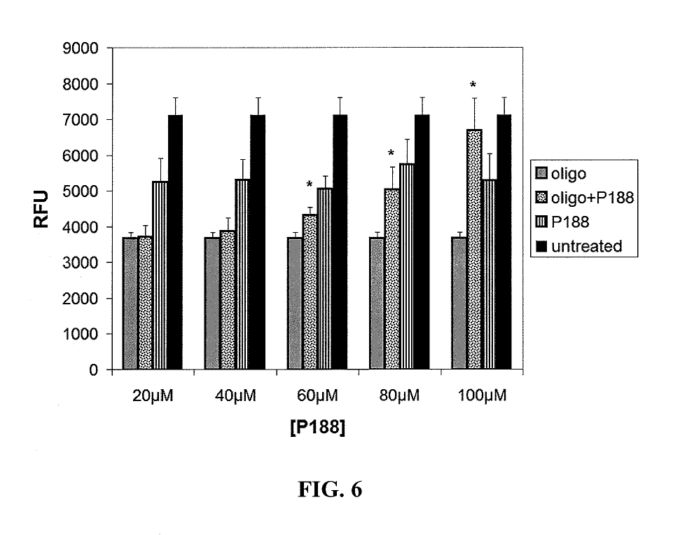

Upon addition of increasing concentrations of P188 to membrane-damaged cells, more P188 molecules plug up the leaky membranes and increase the cells' capabilities to employ their natural exocytic vesicle fusion repair mechanism to reseal the plasma membrane. This attempt to patch up the damaged plasma membrane likely increases the cell's metabolism, and would be indicated by increased reduction of the AlamarBlue reagent. Though cell viability remained relatively invariant with increasing concentrations of P188 alone in (FIG. 6), the health of oligomer and P188-treated cells does consistently and significantly increase with increasing P188 concentration, and even surpasses that of P188-only treated cells.

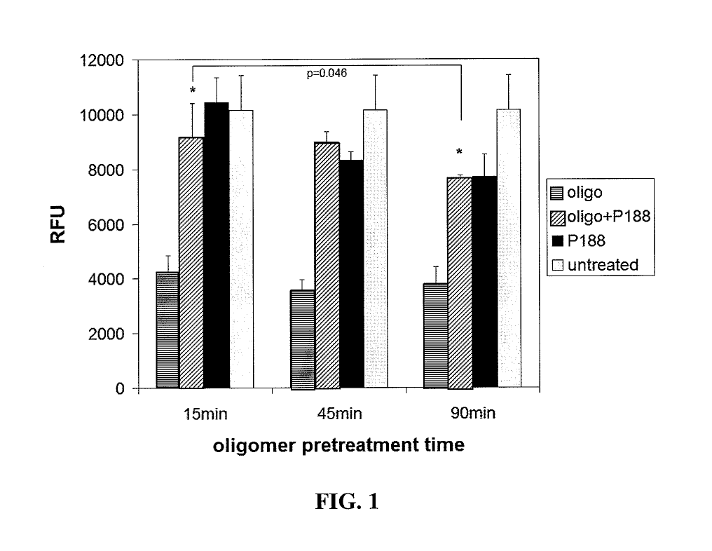

The apparent reduction in cell viability of cells treated with P188 alone is not statistically significant compared to untreated controls (FIG. 1 and FIG. 6), and can be attributed to the reduced intracellular energy metabolism as a result of prolonged exposure to poloxamer⁴⁸. Additionally, prolonged exposure to high concentrations of poloxamers has been reported to cause poor cell growth and reduced protective effects in a cell-line specific manner^{48; 49; 50}. The AlamarBlue reagent is therefore sensitive to this resultant decreased bioreduction rate.

Our results are consistent with the interpretation that P188 inserts into oligomer-damaged membranes and temporarily increases local lipid packing density, thereby plugging the defects and preventing the uncontrolled flux of ions and cellular contents. Subsequently, the cell's natural calcium-triggered exocytic vesicle fusion repair mechanism works to reseal the plasma membrane, restoring surface pressure and lipid density so that P188 can no longer maintain its position and is eventually squeezed out of the membrane^{33; 43}. In this way, the pro-apoptotic signals are minimized and the cell has a better chance at survival. Since P188 is non-toxic and approved for human therapeutic application, it may represent a potential therapeutic against neuronal membrane damage caused by oligomer interaction with cell membranes.

### I. Poloxamers

Poloxamers are nonionic triblock copolymers composed of a central hydrophobic chain of polyoxypropylene (poly(propylene oxide)) flanked by two hydrophilic chains of polyoxyethylene (poly(ethylene oxide)). The word “poloxamer” was coined by Irving Schmolka. Poloxamers are also known by the trade name PLURONICS™. Because the lengths of the polymer blocks can be customized, many different poloxamers exist that have slightly different properties. For the generic term “poloxamer”, these copolymers are commonly named with the letter “P” (for poloxamer) followed by three digits, the first two digits×100 give the approximate molecular mass of the polyoxypropylene core, and the last digit×10 gives the percentage polyoxyethylene content (e.g., P407=Poloxamer with a polyoxypropylene molecular mass of 4,000 g/mol and a 70% polyoxyethylene content). For the Pluronic tradename, coding of these copolymers starts with a letter to define its physical form at room temperature (L=liquid, P=paste, F=flake (solid)) followed by two or three digits. The first digit (two digits in a three-digit number) in the numerical designation, multiplied by 300, indicates the approximate molecular weight of the hydrophobe; and the last digit×10 gives the percentage polyoxyethylene content (e.g., L61=Pluronic with a polyoxypropylene molecular mass of 1,800 g/mol and a 10% polyoxyethylene content). In the example given, poloxamer 181 (P181)=Pluronic L61.

Because of their amphiphilic structure, the polymers have surfactant properties that make them useful in industrial applications. Among other things, they can be used to increase the water solubility of hydrophobic, oily substances or otherwise increase the miscibility of two substances with different hydrophobicities. For this reason, these polymers are commonly used in industrial applications, cosmetics, and phaiinaceuticals. They have also been used as model systems for drug delivery applications. In bioprocess applications, pluronic is also used in cell culture media for its cell cushioning effects because its addition leads to less stressful shear conditions for cells in reactors.

A poloxamer comprises an ethylene oxide-propylene oxide block copolymer, which preferably has the structure (PEG)ₓ-(PPG)_{y}-(PEG)_{z}, where x, y and z are integers and x is usually equal to z.

### II. Protein Misfolding Disorders

In the present invention, protein misfolding disorders including, but not limited to Alzheimer's Disease are treated using the treatment methods disclosed herein.

A. Amyloid

Amyloids are insoluble fibrous protein aggregates sharing specific structural traits. Abnormal accumulation of amyloid in organs may lead to amyloidosis, and may play a role in various other neurodegenerative diseases. The classical, histopathological definition of amyloid is an extracellular, proteinaceous deposit exhibiting beta sheet structure. Common to most cross-beta type structures they are generally identified by apple-green birefringence when stained with congo red and seen under polarized light. These deposits often recruit various sugars and other components such as Serum Amyloid P component, resulting in complex, and sometimes inhomogeneous structures. A more recent, biophysical definition is broader, including any polypeptide which polymerizes to form a cross-beta structure, in vivo, or in vitro. Some of these, although demonstrably cross-beta sheet, do not show some classic histopathological characteristics such as the Congo red birefringence.

Amyloid is characterized by a cross-beta sheet quaternary structure; that is, the beta-strands of the stacked beta-sheets come from different protein monomers and align perpendicular to the axis of the fibril. While amyloid is usually identified using fluorescent dyes, stain polarimetry, circular dichroism, or FTIR (all indirect measurements), the “gold-standard” test to see if a structure contains cross-beta fibers is by placing a sample in an X-ray diffraction beam. There are two characteristic scattering diffraction signals produced at 4.7 and 10 Ångstroms (0.47 nm and 1.0 nm), corresponding to the interstrand and stacking distances in beta sheets. It should be noted that the “stacks” of beta sheet are short and traverse the breadth of the amyloid fibril; the length of the amyloid fibril is built by aligned strands.

For these peptides, cross-polymerization (fibrils of one polypeptide sequence causing other fibrils of another sequence to form) is observed in vitro and possibly in vivo. This phenomenon is important since it would explain interspecies prion propagation and differential rates of prion propagation, as well as a statistical link between Alzheimer's and type 2 diabetes. In general, the more similar the peptide sequence the more efficient cross-polymerization is, though entirely dissimilar sequences can cross-polymerize and highly similar sequences can even be “blockers” which prevent polymerization. Polypeptides will not cross-polymerize their mirror-image counterparts, indicating that the phenomenon involves specific binding and recognition events.

B. Alzheimer's Disease

Alzheimer's Disease (AD), the most common cause of late life dementia in the developed world, is characterized by the cerebral accumulation of misfolded and aggregated amyloid-β peptide (Aβ). This incurable, degenerative, and terminal disease was first described by German psychiatrist Alois Alzheimer in 1906. Generally it is diagnosed in people over 65 years of age, although the less-prevalent early-onset Alzheimer's can occur much earlier. An estimated 26.6 million people worldwide had Alzheimer's in 2006; this number may quadruple by 2050.

Although each sufferer experiences Alzheimer's in a unique way, there are many common symptoms. The earliest observable symptoms are often mistakenly thought to be ‘age-related’ concerns, or manifestations of stress. In the early stages, the most commonly recognized symptom is memory loss, such as difficulty in remembering recently learned facts. When a doctor or physician has been notified, and AD is suspected, the diagnosis is usually confirmed with behavioral assessments and cognitive tests, often followed by a brain scan if available. As the disease advances, symptoms include confusion, irritability and aggression, mood swings, language breakdown, long-term memory loss, and the general withdrawal of the sufferer as their senses decline. Gradually, bodily functions are lost, ultimately leading to death. Individual prognosis is difficult to assess, as the duration of the disease varies. AD develops for an indeterminate period of time before becoming fully apparent, and it can progress undiagnosed for years. The mean life expectancy following diagnosis is approximately seven years. Fewer than three percent of individuals live more than fourteen years after diagnosis.

The cause and progression of Alzheimer's disease are not well understood. Research indicates that the disease is associated with plaques and tangles in the brain. Currently-used treatments offer a small symptomatic benefit; no treatments to delay or halt the progression of the disease are as yet available. As of 2008, more than 500 clinical trials were investigating possible treatments for AD, but it is unknown if any of them will prove successful. Many measures have been suggested for the prevention of Alzheimer's disease, but their value is unproven in slowing the course and reducing the severity of the disease. Mental stimulation, exercise, and a balanced diet are often recommended, as both a possible prevention and a sensible way of managing the disease.

C. Diagnosis of Alzheimer's Disease

Alzheimer's disease is usually diagnosed clinically from the patient history, collateral history from relatives, and clinical observations, based on the presence of characteristic neurological and neuropsychological features and the absence of alternative conditions. Advanced medical imaging with computed tomography (CT) or magnetic resonance imaging (MRI), and with single photon emission computed tomography (SPECT) or positron emission tomography (PET) can be used to help exclude other cerebral pathology or subtypes of dementia. Assessment of intellectual functioning including memory testing can further characterize the state of the disease. Medical organizations have created diagnostic criteria to ease and standardize the diagnostic process for practicing physicians. Sometimes the diagnosis can be confirmed or made at post-mortem when brain material is available and can be examined histologically.

The National Institute of Neurological and Communicative Disorders and Stroke and the Alzheimer's Disease and Related Disorders Association (now known as the Alzheimer's Association) established the most commonly used diagnostic criteria for Alzheimer's disease. These criteria require that the presence of cognitive impairment, and a suspected dementia syndrome, be confirmed by neuropsychological testing for a clinical diagnosis of possible or probable AD. A histopathologic confirmation including a microscopic examination of brain tissue is required for a definitive diagnosis. Good statistical reliability and validity have been shown between the diagnostic criteria and definitive histopathological confirmation. Eight cognitive domains are most commonly impaired in AD—memory, language, perceptual skills, attention, constructive abilities, orientation, problem solving and functional abilities. These domains are equivalent to the NINCDS-ADRDA Alzheimer's Criteria as listed in the Diagnostic and Statistical Manual of Mental Disorders (DSM-IV-TR) published by the American Psychiatric Association.

Neuropsychological screening tests can help in the diagnosis of AD. In them patients have to copy drawings similar to the one shown in the picture, remember words, read or sum. Neuropsychological tests such as the mini-mental state examination (MMSE), are widely used to evaluate the cognitive impairments needed for diagnosis. More comprehensive test arrays are necessary for high reliability of results, particularly in the earliest stages of the disease. Neurological examination in early AD will usually provide normal results, except for obvious cognitive impairment, which may not differ from standard dementia. Further neurological examinations are crucial in the differential diagnosis of AD and other diseases. Interviews with family members are also utilized in the assessment of the disease. Caregivers can supply important information on the daily living abilities, as well as on the decrease, over time, of the person's mental function. A caregiver's viewpoint is particularly important, since a person with AD is commonly unaware of his own deficits. Many times, families also have difficulties in the detection of initial dementia symptoms and may not communicate accurate information to a physician. Supplemental testing provides extra information on some features of the disease or is used to rule out other diagnoses. Blood tests can identify other causes for dementia than AD—causes which may, in rare cases, be reversible. Psychological tests for depression are employed, since depression can either be concurrent with AD or be the cause of cognitive impairment.

When available as a diagnostic tool, SPECT and PET neuroimaging are used to confirm a diagnosis of Alzheimer's in conjunction with evaluations involving mental status examination. The ability of SPECT to differentiate Alzheimer's disease from other possible causes in somebody already known to be suffering from dementia, appears to be superior to attempts to diagnose by mental testing and history. A new technique known as PiB PET has been developed for directly and clearly imaging beta-amyloid deposits in vivo using a tracer that binds selectively to the Abeta deposits. Another recent objective marker of the disease is the analysis of cerebrospinal fluid for amyloid beta or tau proteins. Both advances have led to the proposal of new diagnostic criteria.

D. Treatment of Protein Misfolding Disorders

The present invention is focused on providing novel treatment methods and compositions for protein misfolding or aggregation disorders, such as Alzheimer's disease. As described previously, the effects of aggregation could potentially be treated and reversed by membrane sealing polymers.

AD belongs to a group of diseases in which the hallmark event is the misfolding, aggregation and tissue accumulation of proteins. These diseases known as Protein Misfolding Disorders (PMDs) include Alzheimer's disease (AD), Parkinson's disease (PD), amyotrophic lateral sclerosis (ALS), Huntington's disease (HD), Diabetes type-2, systemic amyloidosis and prion diseases, among more than 15 others (Chiti and Dobson, 2006; Soto, 2001; Carrell, 2005). A list of the diseases and proteins involved in PMDs is shown in Table 1.

It has been suggested that tissue deposition of misfolded protein aggregates might be responsible for the activation of cell impairment and death, leading subsequently to clinical symptoms in affected individuals. The insidious clinical symptoms, the progressive nature of the illness and the lack of efficient therapeutic treatments for these diseases lead to severe problems for the quality of life of affected people and their families in both, social and economic aspects. The expenses for the treatment and care of patients are very high and these costs progressively increase due to the severity of the disease. In addition, it is expected that the number of people that will be affected by these maladies will increase at a high rate during the coming years. Moreover, the lack of early diagnostic methods or effective treatments paints a bleak scenario for the future.

In spite of the important differences in clinical manifestation, PMDs share some common features such as their appearance late in life, the progressive and chronic nature of the disease and the presence of deposits of misfolded protein aggregates (Soto, 2003). These deposits are a typical disease signature and although in each disease the main protein component is different (Table 1), they have similar morphological, structural and staining characteristics. Amyloid is the name originally given to extracellular protein deposits found in AD and systemic amyloid disorders (Glenner, 1980), but it is nowadays used to refer in general to disease-associated protein aggregates (Soto, 2003). All protein aggregates share similar structural and biochemical characteristics. Structurally, misfolding of proteins increases the level of β-sheet structure, leading to the formation of amyloid polymers organized as cross-β structures (Blake et al., 1996). As a consequence, protein aggregates are resistant to proteolysis, denaturation and general cellular clearance mechanisms.

The cellular factors and processes leading to the misfolded conformation have been partially identified. Among them, several mutations destabilizing the folded conformation and promoting its shift to the misfolded form have been identified in each protein (Hardy and Gwinn-Hardy, 1998). These mutations usually results in dominant inheritance of the diseases. Nevertheless, familial forms of the disease represent a small percentage of the total incidence of PMDs, and the majority of the cases have a sporadic origin. The discovery of mutations has led the development of transgenic animal models over-expressing mutated proteins, where aggregates accumulation and cellular impairments occur in a similar form as in the human disease (Rockenstein et al., 2007).

In spite of the key role of misfolded proteins in the disease, the mechanisms leading to cellular damage and tissue dysfunction are still unclear. The toxicity of protein aggregates has been extensively documented in vitro and in vivo (Canevari et al., 2004; Carrell, 2005; Haass and Selkoe, 2007). However it is still not clear which type of aggregates are the most toxic species. Recent evidence supports the hypothesis that smaller and soluble oligomeric aggregates on pathway to form the large fibrillar deposits could be the molecules mostly responsible for the toxic effects observed in these maladies (Haass and Selkoe, 2007; Glabe, 2006; Caughey and Lansbury, 2003).

TABLE 1
List of some Protein Misfolding Disorders, the protein implicated
and the organ mostly affected by deposition of aggregates.

| | PROTEIN | AFFECTED |
|---|---|---|
| DISEASES | INVOLVED | ORGAN |
| Alzheimer's disease | Amyloid-β | Brain |
| | protein, Tau | |
| Type II diabetes | Islet amyloid | Pancreas |
| | polypeptide | |
| Parkinson's disease | α-synuclein | Brain |
| Primary amyloidosis | Immunoglobulin | Mostly kidney, liver, |
| (Implicated in | light chain | heart, nerves |
| Multiple myeloma, | | |
| β-cell dyscracias) | | |
| Huntington's disease | Huntingtin | Brain |
| Secondary or reactive | Amyloid-A | Mostly spleen, liver |
| amyloidosis | | and kidney |
| Spinocerebral ataxias | Ataxins | Brain |
| Transmissible spongiform | Prion protein | Brain |
| encephalopathies | | |
| Hemodialysis-related | β2-microglobulin | Bones and joints |
| amyloidosis | | |
| Amyotrophic lateral | Superoxide | Brain |
| sclerosis | dismutase | |
| Familial dementia of | ABri or ADan | Brain |
| British or Danish type | polypeptides | |
| Senile systemic | Transthyretin | Heart, Kidney, Lungs, |
| amyloidosis, familial | | peripheral nerves |
| amyloid polyneuropathy | | |
| Hereditary cerebral | Cystatin C | Brain |
| hemorrhage with | | |
| amyloidosis | | |
| Icelandic-type | | |
| Familial amyloidosis, | Gelsolin | Peripheral and Central |
| finnish-type | | nervous system |
| Familial amyloid | Apolipoprotein | Mostly in aorta |
| polyneuropathy | A-I | |
| Senile Amyloidosis | Apolipoprotein | Multiple organs |
| | A-II | |
| Hereditary systemic | Lysozyme | Liver, spleen, |
| amyloidosis, familial | | Gastrointestinal |
| visceral amyloidosis | | tract |
| Serpin deficiency | Serpins | Liver, brain |
| disorders (cirrhosis, | | |
| angioedema) | | |

### III. Pharmaceutical Formulation and Administration

Pharmaceutical formulations of the present invention comprise an effective amount of one or more membrane sealing polymer or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical formulation that contains at least one active compound or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The invention may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include one or more compounds represented by formula I, II, III, IV, or V one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the compounds may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, the active compound may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

In further embodiments, the active compound may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compound in a therapeutically effective amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

In certain embodiments, the pharmaceutical compositions may be delivered by intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### IV. Combination Therapy

In order to increase the effectiveness of the active compound or formulation thereof, it may be desirable to combine the compound(s) or formulation with other agents effective in the treatment of Alzheimer's disease or other protein misfolding disorders. More generally, these other compositions would be provided in a combined amount effective to inhibit or reduce cell toxicity. This process may involve treating the subject with the active compound and the agent(s) or multiple factor(s) at the same time. This may be achieved by treating the subject with a single composition or pharmacological formulation that includes both agents, or with two distinct compositions or formulations, at the same time, wherein one composition includes the active compound and the other includes the second agent(s).

In the context of the present invention, it is contemplated that the active compound or formulation could be used similarly in conjunction with a second pharmaceutical or psychosocial intervention. Alternatively, the active compound or formulation may precede or follow the other agent treatment by intervals ranging from minutes to weeks. In embodiments where the other agent and active compound or formulation are applied separately to the subject, one would generally ensure that a significant period of time did not expire between the time of each delivery, such that the agent and active compound or formulation would still be able to exert an advantageously combined effect on amyloid aggregates. In such instances, it is contemplated that one may treat the subject with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, active compound “membrane sealing polymer” or formulation is “A” and the secondary agent, such as a second pharmaceutical or psychosocial intervention, is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

Administration of the therapeutic compound of the present invention to a patient will follow general protocols for the administration of therapeutics, taking into account the toxicity, if any, of the active compound. It is expected that the treatment cycles would be repeated as necessary.

A. Pharmaceutical Intervention

Four medications are currently approved by regulatory agencies such as the U.S. Food and Drug Administration (FDA) and the European Medicines Agency (EMEA) to treat the cognitive manifestations of AD: three are acetylcholinesterase inhibitors and the other is memantine, an NMDA receptor antagonist.

Reduction in the activity of the cholinergic neurons is a well-known feature of Alzheimer's disease. Acetylcholinesterase inhibitors are employed to reduce the rate at which acetylcholine (ACh) is broken down, thereby increasing the concentration of ACh in the brain and combating the loss of ACh caused by the death of cholinergic neurons. As of 2008, the cholinesterase inhibitors approved for the management of AD symptoms are donepezil (brand name Aricept), galantamine (Razadyne), and rivastigmine (branded as Exelon and Exelon Patch). There is evidence for the efficacy of these medications in mild to moderate Alzheimer's disease, and some evidence for their use in the advanced stage. Only donepezil is approved for treatment of advanced AD dementia. The use of these drugs in mild cognitive impairment has not shown any effect in a delay of the onset of AD. The most common side effects are nausea and vomiting, both of which are linked to cholinergic excess. These side effects arise in approximately ten to twenty percent of users and are mild to moderate in severity. Less common secondary effects include muscle cramps, decreased heart rate (bradycardia), decreased appetite and weight, and increased gastric acid production.

Glutamate is a useful excitatory neurotransmitter of the nervous system, although excessive amounts in the brain can lead to cell death through a process called excitotoxicity which consists of the overstimulation of glutamate receptors. Excitotoxicity occurs not only in Alzheimer's disease, but also in other neurological diseases such as Parkinson's disease and multiple sclerosis. Memantine (brand names Akatinol, Axura, Ebixa/Abixa, Memox and Namenda), is a noncompetitive NMDA receptor antagonist first used as an anti-influenza agent. It acts on the glutamatergic system by blocking NMDA receptors and inhibiting their overstimulation by glutamate. Memantine has been shown to be moderately efficacious in the treatment of moderate to severe Alzheimer's disease. Its effects in the initial stages of AD are unknown. Reported adverse events with memantine are infrequent and mild, including hallucinations, confusion, dizziness, headache and fatigue. The combination of memantine and donepezil has been shown to be “of statistically significant but clinically marginal effectiveness”.

Antipsychotic drugs are modestly useful in reducing aggression and psychosis in Alzheimer's patients with behavioral problems, but are associated with serious adverse effects, such as cerebrovascular events, movement difficulties or cognitive decline, that do not permit their routine use. When used in the long-term, they have been shown to associate with increased mortality.

B. Psychosocial Intervention

Psychosocial interventions are used as an adjunct to pharmaceutical treatment and can be classified within behavior-, emotion-, cognition- or stimulation-oriented approaches. Research on efficacy is unavailable and rarely specific to AD, focusing instead on dementia in general.

Behavioral interventions attempt to identify and reduce the antecedents and consequences of problem behaviors. This approach has not shown success in improving overall functioning, but can help to reduce some specific problem behaviors, such as incontinence. There is a lack of high quality data on the effectiveness of these techniques in other behavior problems such as wandering.

Emotion-oriented interventions include reminiscence therapy, validation therapy, supportive psychotherapy, sensory integration, also called snoezelen, and simulated presence therapy. Supportive psychotherapy has received little or no formal scientific study, but some clinicians find it useful in helping mildly impaired patients adjust to their illness. Reminiscence therapy (RT) involves the discussion of past experiences individually or in group, many times with the aid of photographs, household items, music and sound recordings, or other familiar items from the past. Although there are few quality studies on the effectiveness of RT, it may be beneficial for cognition and mood. Simulated presence therapy (SPT) is based on attachment theories and involves playing a recording with voices of the closest relatives of the person with Alzheimer's disease. There is preliminary evidence indicating that SPT may reduce anxiety and challenging behaviors. Finally, validation therapy is based on acceptance of the reality and personal truth of another's experience, while sensory integration is based on exercises aimed to stimulate senses. There is little evidence to support the usefulness of these therapies.

The aim of cognition-oriented treatments, which include reality orientation and cognitive retraining, is the reduction of cognitive deficits. Reality orientation consists in the presentation of information about time, place or person in order to ease the understanding of the person about its surroundings and his or her place in them. On the other hand cognitive retraining tries to improve impaired capacities by exercitation of mental abilities. Both have shown some efficacy improving cognitive capacities, although in some studies these effects were transient and negative effects, such as frustration, have also been reported.

Stimulation-oriented treatments include art, music and pet therapies, exercise, and any other kind of recreational activities. Stimulation has modest support for improving behavior, mood, and, to a lesser extent, function. Nevertheless, as important as these effects are, the main support for the use of stimulation therapies is the improvement in the person's daily life routines.

### Examples

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1

### Poloxamer Increases Cell Survival

P188 increases the viability of oligomer-treated cells in a time-dependent manner Poloxamer P188 treated SH-SY5Y cells demonstrated increased survival following incubation with Aβ42 oligomers (FIG. 1). Importantly, the earlier P188 was applied to the cells, the more likely they were to recover. When P188 was added to cells 15 min post-oligomer exposure, there was a 16% increase in cell survival as compared to those that were exposed to oligomers for 90 minutes prior to P188 addition (FIG. 1). Therefore, the longer the cells were exposed to the oligomers prior to P188 addition, the less effectively P188 rescued the damaged cells, as assayed by AlamarBlue fluorescence.

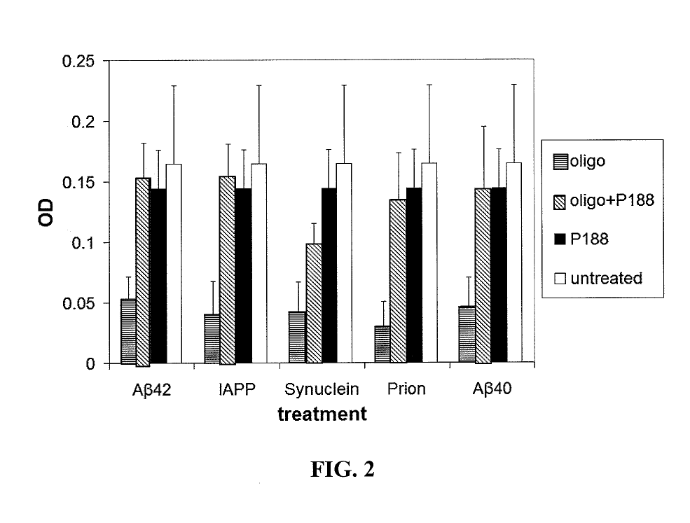

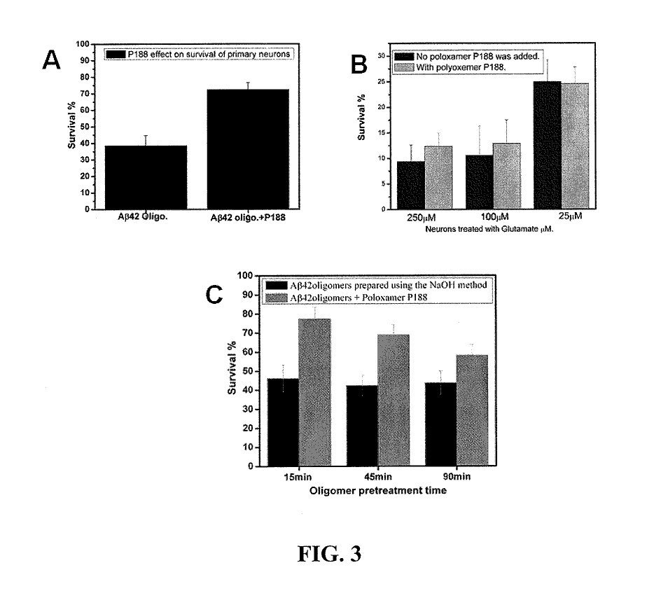

Similarly for oligomers of other amyloid proteins, addition of P188 after 15 minutes increased SH-SY5Y cell survival by 61% (Aβ42), 69% (IAPP), 34% (α-synuclein), 64% (prion 106-126), and 59% (Aβ40), compared to oligomer treatment alone as measured by MTT (FIG. 2). Additionally, application of P188 to primary rat hippocampal neurons treated with Aβ42 oligomers, nearly doubled the percentage of surviving neurons, (FIG. 3A). We also showed that the protective effect of P188 in neuronal cultures is specific to membrane damage and not a general rescue from any toxic insult, such as glutamate toxicity. The inventors found that neurons treated with glutamate do not exhibit increased survival following application of P188 (FIG. 3B), as measured by MTT.

Many methods were reported for oligomers preparation, here it is shown that P188 rescues the toxicity induced by Aβ42 oligomers prepared by two different methods the HFIP-based method^{6; 34} (FIG. 1) and the NaOH-based method described previously³⁵ (FIG. 3C). Moreover, it is shown that the rescue by P188 does not depend on its interaction with and disruption of the oligomers themselves, oligomers toxic conformation is preserved in the presence of P188 (FIG. 4).

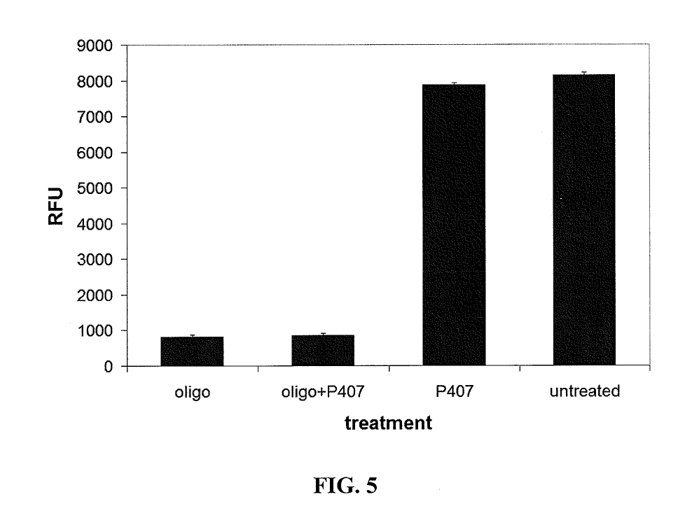

Effect of P188 on cell survival is specific and concentration-dependent Using the nonspecific poloxamer 407 (P407), it was confirmed that the rescuing effects of P188 were specific (FIG. 5). Unlike P188 which has membrane sealing properties, P407 is typically used as an inactive ingredient that assists in the preparation, formulation, and stabilization of poorly water-soluble molecules³⁶. SH-SY5Y cells treated with 2.5 μM Aβ42 oligomers and subsequently with 20 ng/μl P407 did not show any significant improvement in cell survival as assayed by AlamarBlue, although P407 on its own was non-toxic to the cells, compared to the untreated controls.

To determine the range of effectiveness of P188, the inventors studied a range of concentrations, from 8.5-42 ng/μl (˜20-100 μM). SH-SY5Y cells were treated with 2.5 μM Aβ42 oligomers for 15 min after which P188 was added to the wells. P188 was significantly effective at improving cell viability at 25.5 ng/μl (p=0.05) and most effective at 42 ng/μl (p=0.02) (FIG. 6).

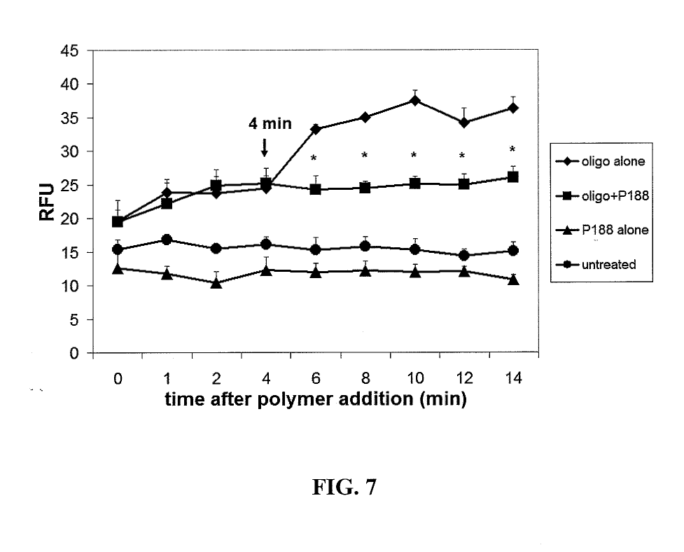

P188 blocks dye leakage into cells permeabilized by oligomer treatment To demonstrate that the rescue of oligomer toxicity observed in cells by P188 was specifically due to reversal of the oligomer-induced membrane damage, the inventors measured the leakage of EthD-1 into cells treated with Aβ42 oligomers and immediately after P188. Almost immediately after adding oligomers, the cell membranes were leaky, and continued to take up EthD-1, as indicated by the increase in fluorescence (FIG. 7). However, within 4 min of adding P188, the dye leakage stopped, and remained steady over a period of 14 min (FIG. 7).

### Materials and Methods

Preparation of oligomers and polymer Oligomers were prepared from Aβ, IAPP, synuclein and prion 106-126 as described previously^{6; 34}. Briefly, peptides (0.3 mg) were dissolved in hexafluoroisopropanol (HFIP) for 10-20 min at room temperature. Following incubation, the samples were centrifuged for 15 min at 14,000×G and the supernatant fraction (pH 2.8-3.5) was transferred to a new siliconized tube and subjected to a gentle stream of N₂ for 10 min to evaporate the HFIP. The samples were then stirred at 500 rpm using a Teflon-coated micro stir bar for 24-48 h at 22° C. The final peptide concentration was 0.3 mg/ml.

In addition to the HFIP-based oligomers preparations, Aβ oligomers were also prepared by the NaOH-based method described previously³⁵. Briefly, Aβ₄₂ stock solutions (2 mM) were obtained by dissolving the lyophilized peptide in 100 mM NaOH followed by water bath sonication for 30 s. The oligomerization reaction was initiated by diluting the stock solution in phosphate buffered saline (PBS), pH=7.4, 0.02% sodium azide (45 μM final Aβ₄₂ concentration, final pH=7.4). The solutions were allowed to oligomerize for 3 days at room temperature. Oligomers formation was checked by dot blot and ELISA using A11.

Poloxamer 188 and 407 (BASF, Parsippany, N.J.) were prepared at a concentration of 200 μg/ml by dissolving in doubly distilled water.

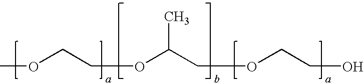

| Poloxamer | a | b |
|---|---|---|
| 188 | 80 | 27 |
| 407 | 101 | 56 |

Cell culture SH-SY5Y human neuroblastoma cells were maintained in Dulbecco's modified Eagle's medium (DMEM) (Gibco) supplemented with 10% fetal bovine serum, glutamine (4 mM), penicillin (200 unit/ml), streptomycin (200 μg/ml), and sodium pyruvate (1 μM). Cells were maintained at 37° C. in 5% CO₂ and the medium was replaced every 2 days. Cells (˜15,000) were plated in 96-well plates (Corning) and grown overnight.

For primary neurons, cerebral cortices were dissected out from E18 Sprague-Dawley rat brains and exposed to 0.125% of trypsin in Hank's balanced salt solution free of calcium and magnesium (CMF) for 7 min at 37° C. Cortical tissues were then resuspended in serum-free neurobasal medium supplemented with N₂ (NB/N₂), and dissociated by trituration using flame polished siliconized Pasteur pipettes. Viable cells, quantified by trypan blue exclusion, were plated at 5×10⁴ cells per well in 0.5 mL NB/N₂ medium (i.e. 250 cells/mm²) in poly 1-lysine (10 μg/mL) coated 24-well plates (Costar, Cambridge, Mass., USA) and grown 3-4 days in vitro before treatments.

Cell viability assays MTT assay: Cell viability was assessed spectrophotometrically using an MTT (3-[4,5-dimethylthiazol-2-yl]-2,5-diphenyl tetrazolium bromide)-based assay (Sigma-Aldrich, St. Louis, Mo.). The conditions were carried out in triplicate. After four hours, MTT dissolved in DMEM as added to the cells and incubated for an additional four hours. Insoluble crystals were dissolved by adding MTT solubilization solution (10% Triton X-100, 0.1 N HCl in anhydrous isopropanol) and absorbance was measured at 570 nm.

AlamarBlue assay SH-SY5Y human neuroblastoma cells and RPE human fetal retinal pigment epithelial cells were grown to confluence in 96-well plates. Cells were pre-treated with 2.5 μM oligomer followed by P188, 15, 45, and 90 minutes later, at a final concentration of 20 ng/μl. Untreated controls, as well as cells treated with polymer or 2.5 μM oligomer alone were also included. Cytotoxicity was measured using the AlamarBlue assay kit (Serotec), which measures metabolic reduction of the AlamarBlue reagent as an indicator of cell survival and proliferation. AlamarBlue reagent was added in an amount equal to 10% of the culture volume, and cultures were returned to the incubator. Fluorescence was measured 3.5 h later with excitation wavelength of 545 nm and emission wavelength of 590 nm using a fluorescence plate reader (GeminiXPS, Molecular Devices). All measurements were performed in triplicate.

Dye leakage assay Ethidium homodimer-1 (EthD-1) fluoresces bright red upon entering the nuclei of damaged cells and binding DNA and is excluded from intact membranes. EthD-1 (Molecular Probes) was added to cell media at a final concentration of 4 μM and allowed to stabilize for about 5 min before taking an initial fluorescence reading using a fluorescence plate reader (ex: 530 nm, em: 645 nm). Once the initial reading was collected, Aβ42 oligomers were applied to the cells at a final concentration of ˜2.5 μM, and 20 ng/μl of P188 was added 1 min later. Fluorescence was measured every 2 min thereafter for about 14 minutes.

Oligomers dot blot analysis Oligomers from a variety of amyloid proteins were incubated with excess P188 (1:2, protein: polymer, by mass), in the presence or absence of cell media, for 30 min and 1 h. Next, 2 μl of each sample was applied to a nitrocellulose membrane, blocked in 10% non-fat milk in Tris-buffered saline (TBS) containing 0.01% Tween 20 (TBS-T), at room temperature for 1 h (or overnight at 4° C.), washed three times for 5 min each with TBS-T and incubated for 1 hr at room temperature anti-oligomer antibody (A11) (0.1 μg/ml in 5% non-fat milk in TBS-T) for 1 h. The membranes was washed three times for 5 min each with TBS-T, incubated with horseradish peroxidase conjugated anti-rabbit IgG (Promega) diluted 1:10,000 in 5% non-fat milk/TBS-T and incubated for 1 hour at room temperature. The blot was washed three times with TBS-T and developed with ECL chemiluminescence kit from Amersham-Pharmacia (Piscataway, N.J.). Films we scanned an the signal was quantitated using Scion Imaging Software.

Oligomers ELISA analysis 10 μl aliquots of each sample were applied to 96-well clear, flat bottom plates containing 100 μl coating buffer (0.1 M sodium bicarbonate, pH=9.6). The plates were incubated for 1 h at 37° C., washed one time with TBS-T, then blocked with 200 μl of 10% non-fat milk in TBS-T for 1 h at 37° C., and washed three times. Then, 100 μl of A11 antibody (0.1 μg/ml in 5% non-fat milk in TBS-T) was added to each well and the plates were incubated for 1 h at 37° C., washed three times with TBS-T. 100 μl of HRP-conjugated anti-rabbit IgG (1:10,000 dilution in 5% non-fat milk/TBS-T) were added to each well and the plates were incubated for 1 h at 37° C. The plates were then washed three times with TBS-T and developed with 3,3′,5,5′-tetramethylbenzidine (TMB). The reactions were stopped with 100 μl of 1 M HCl and measured at 450 nm. Samples were measured in triplicate.

Oligomers western blot analysis 10 μl aliquots of each oligomer samples were mixed with 10 μl 2×SDS sample buffer, and loaded onto 4-20% Tris-HCl gels, samples were not boiled to avoid any disassociation of the oligomers. The gels were transferred to nitrocellulose membranes which were then subjected to the same treatment as described for the dot blot analysis using A11 antibody.

### REFERENCES

The following references, to the extent that they provide exemplary procedural or other details supplementary to those set forth herein, are specifically incorporated herein by reference.
1. Bucciantini, M., Giannoni, E., Chiti, F., Baroni, F., Formigli, L., Zurdo, J. S., Taddei, N., Ramponi, G., Dobson, C. M. & Stefani, M. (2002). Inherent toxicity of aggregates implies a common mechanism for protein misfolding diseases. Nature 416, 507-511.
2. Caughey, B. & Lansbury, P. T. (2003). Protofibrils, pores, fibrils, and neurodegeneration: separating the responsible protein aggregates from the innocent bystanders. Annu Rev Neurosci 26, 267-98.
3. Klein, W. L., Stine, W. B., Jr. & Teplow, D. B. (2004). Small assemblies of unmodified amyloid beta-protein are the proximate neurotoxin in Alzheimer's disease. Neurobiol Aging 25, 569-80.
4. Stefani, M. & Dobson, C. M. (2003). Protein aggregation and aggregate toxicity: new insights into protein folding, misfolding diseases and biological evolution. J Mol Med 81, 678-99.
5. Walsh, D. M. & Selkoe, D. J. (2004). Oligomers on the brain: the emerging role of soluble protein aggregates in neurodegeneration. Protein Pept Lett 11, 213-28.
6. Demuro, A., Mina, E., Kayed, R., Milton, S. C., Parker, I. & Glabe, C. G. (2005). Calcium Dysregulation and Membrane Disruption as a Ubiquitous Neurotoxic Mechanism of Soluble Amyloid Oligomers. J. Biol. Chem. 280, 17294-17300.
7. Porat, Y., Kolusheva, S., Jelinek, R. & Gazit, E. (2003). The human islet amyloid polypeptide forms transient membrane-active prefibrillar assemblies. Biochemistry 42, 10971-7.
8. Kayed, R., Sokolov, Y., Edmonds, B., McIntire, T. M., Milton, S. C., Hall, J. E. & Glabe, C. G. (2004). Permeabilization of lipid bilayers is a common conformation-dependent activity of soluble amyloid oligomers in protein misfolding diseases. J Biol Chem 279, 46363-6.
9. Canale, C., Torrassa, S., Rispoli, P., Relini, A., Rolandi, R., Bucciantini, M., Stefani, M. & Gliozzi, A. (2006). Natively Folded HypF-N and Its Early Amyloid Aggregates Interact with Phospholipid Monolayers and Destabilize Supported Phospholipid Bilayers. Biophys. J. 91, 4575-4588.
10. Mattson, M. P., Cheng, B., Davis, D., Bryant, K., Lieberburg, I. & Rydel, R. E. (1992). beta-Amyloid peptides destabilize calcium homeostasis and render human cortical neurons vulnerable to excitotoxicity. J. Neurosci. 12, 376-389.
11. Mattson, M. P. (1994). Calcium and neuronal injury in Alzheimer's disease.

Contributions of beta-amyloid precursor protein mismetabolism, free radicals, and metabolic compromise. Ann. N. Y. Acad. Sci. 747, 50-76.
12. Arispe, N., Pollard, H. B. & Rojas, E. (1994). beta-Amyloid Ca(2+)-channel hypothesis for neuronal death in alzheimer disease. Mol. Cell. Biochem. 140, 119-125.
13. Arispe, N., Pollard, H. B. & Rojas, E. (1993). Giant Multilevel Cation Channels Formed by Alzheimer Disease Amyloid {beta}-Protein [A {beta}P-(1-40)] in Bilayer Membranes. PNAS 90, 10573-10577.
14. Mirzabekov, T., Lin, M. C., Yuan, W. L., Marshall, P. J., Carman, M., Tomaselli, K., Lieberburg, I. & Kagan, B. L. (1994). Channel formation in planar lipid bilayers by a neurotoxic fragment of the beta-amyloid peptide. Biochem. Biophys. Res. Commun. 202, 1142-1148.
15. Lashuel, H. A., Hartley, D., Petre, B. M., Walz, T. & Lansbury, P. T., Jr. (2002). Neurodegenerative disease: amyloid pores from pathogenic mutations. Nature 418, 291.
16. Hirakura, Y., Yiu, W. W., Yamamoto, A. & Kagan, B. L. (2000). Amyloid peptide channels: blockade by zinc and inhibition by Congo red (amyloid channel block). Amyloid 7, 194-9.
17. Mirzabekov, T. A., Lin, M.-c. & Kagan, B. L. (1996). Pore Formation by the Cytotoxic Islet Amyloid Peptide Amylin. J. Biol. Chem. 271, 1988-1992.
18. Kagan, B. L., Azimov, R. H., Azimova, R. (2004). Amyloid peptide channels. J. Membr Biol 202, 1-10.
19. Volles, M. J., Lee, S. J., Rochet, J. C., Shtilerman, M. D., Ding, T. T., Kessler, J. C. & Lansbury, P. T. (2001). Vesicle Permeabilization by Protofibrillar alpha-Synuclein: Implications for the Pathogenesis and Treatment of Parkinson's Disease. Biochemistry 40, 7812-7819.
20. Green, J. D., Kreplak, L., Goldsbury, C., Li Blatter, X., Stolz, M., Cooper, G. S., Seelig, A., Kistler, J. & Aebi, U. (2004). Atomic Force Microscopy Reveals Defects Within Mica Supported Lipid Bilayers Induced by the Amyloidogenic Human Amylin Peptide. Journal of Molecular Biology 342, 877-887.
21. Valincius, G., Heinrich, F., Budvytyte, R., Vanderah, D. J., McGillivray, D. J., Sokolov, Y., Hall, J. E. & Losche, M. (2008). Soluble amyloid beta-oligomers affect dielectric membrane properties by bilayer insertion and domain formation: implications for cell toxicity. Biophys J 95, 4845-61.
22. Sokolov, Y., Kozak, J. A., Kayed, R., Chanturiya, A., Glabe, C. & Hall, J. E. (2006). Soluble amyloid oligomers increase bilayer conductance by altering dielectric structure. J Gen Physiol 128, 637-47.
23. Hannig, J., Zhang, D., Canaday, D. J., Beckett, M. A., Astumian, R. D., Weichselbaum, R. R. & Lee, R. C. (2000). Surfactant Sealing of Membranes Permeabilized by Ionizing Radiation. Radiation Research 154, 171-177.
24. Tung, L., Troiano, G. C., Sharma, V., Raphael, R. M. & Stebe, K. J. (1999). Changes in Electroporation Thresholds of Lipid Membranes by Surfactants and Peptides. Ann NY Acad Sci 888, 249-265.
25. Lee, R. C., Hannig, J., Matthews, K. L., Myerov, A. & Chen, C.-T. (1999). Pharmaceutical Therapies for Sealing of Permeabilized Cell Membranes in Electrical Injuries. Ann NY Acad Sci 888, 266-273.
26. Watanabe, M. & Okada, T. (2003). Lysophosphatidylcholine-induced myocardial damage is inhibited by pretreatment with poloxamer 188 in isolated rat heart. Molecular and Cellular Biochemistry 248, 209-215.
27. Serbest, G., Horwitz, J. & Barbee, K. (2005). The effect of poloxamer-188 on neuronal cell recovery from mechanical injury. J Neurotrauma 22, 119-32.
28. Sheldon, S. S., R. A. Steinhardt. (2005). The mechanisms of cell membrane resealing in rabbit corneal epithelial cells. Curr. Eye Res. 30, 543-554.
29. Frim, D. M., Wright, D. A., Curry, D. J., Cromie, W., Lee, R. & Kang, U. J. (2004). The surfactant poloxamer-188 protects against glutamate toxicity in the rat brain. Neuroreport 15, 171-4.
30. Clarke, M. S., Prendergast, M. A. & Terry, A. V., Jr. (1999). Plasma membrane ordering agent pluronic F-68 (PF-68) reduces neurotransmitter uptake and release and produces learning and memory deficits in rats. Learn Mem 6, 634-49.
31. Schmolka, I. R. (1994). Physical basis for poloxamer interactions. Ann NY Acad Sci 720, 92-97.
32. Prior, S., Gander, B., Lecaroz, C., Irache, J. M. & Gamazo, C. (2004). Gentamicin-loaded microspheres for reducing the intracellular Brucella abortus load in infected monocytes. J. Antimicrob. Chemother. 53, 981-988.
33. Maskarinec, S. A., Hannig, J., Lee, R. C. & Lee, K. Y. (2002). Direct observation of poloxamer 188 insertion into lipid monolayers. Biophys J 82, 1453-9.
34. Kayed, R., Head, E., Thompson, J. L., McIntire, T. M., Milton, S. C., Cotman, C. W. & Glabe, C. G. (2003). Common Structure of Soluble Amyloid Oligomers Implies Common Mechanism of Pathogenesis. Science 300, 486-489.
35. Necula, M., Kayed, R., Milton, S. & Glabe, C. G. (2007). Small molecule inhibitors of aggregation indicate that amyloid beta oligomerization and fibrillization pathways are independent and distinct. J Biol Chem 282, 10311-24.
36. Dumortier, G., Grossiord, J. L., Agnely, F. & Chaumeil, J. C. (2006). A review of poloxamer 407 pharmaceutical and pharmacological characteristics. Pharm Res 23, 2709-28.
37. Kawahara, M., Arispe, N., Kuroda, Y. & Rojas, E. (1997). Alzheimer's disease amyloid beta-protein forms Zn(2+)-sensitive, cation-selective channels across excised membrane patches from hypothalamic neurons. Biophys. J. 73, 67-75.
38. Janson, J., Ashley, R. H., Harrison, D., McIntyre, S. & Butler, P. C. (1999). The mechanism of islet amyloid polypeptide toxicity is membrane disruption by intermediate-sized toxic amyloid particles. Diabetes 48, 491-498.
39. Ashley, R., Harroun, T., Hauss, T., Breen, K. & Bradshaw, J. (2006). Autoinsertion of soluble oligomers of Alzheimer's Abeta(1-42) peptide into cholesterol-containing membranes is accompanied by relocation of the sterol towards the bilayer surface. BMC Structural Biology 6, 21.
40. Yoshiike, Y., Kayed, R., Milton, S. C., Takashima, A. & Glabe, C. G. (2007). Pore-forming proteins share structural and functional homology with amyloid oligomers. Neuromolecular Med 9 (3), 270-5.
41. Hannig, J., Yu, J., Beckett, M., Weichselbaum, R. & Lee, R. C. (1999). Poloxamine 1107 sealing of radiopermeabilized erythrocyte membranes. Int J Radiat Biol 75, 379-85.
42. Sharma, V., Stebe, K., Murphy, J. C. & Tung, L. (1996). Poloxamer 188 decreases susceptibility of artificial lipid membranes to electroporation. Biophys. J. 71, 3229-3241.
43. Guohui, W., Jaroslaw, M., Canay, E., Kristian, K., Markus Jan, W. & Ka Yee, C. L. (2004). Lipid Corralling and Poloxamer Squeeze-Out in Membranes. Physical Review Letters 93, 028101.
44. Reddy, A., Caler, E. V. & Andrews, N. W. (2001). Plasma Membrane Repair Is Mediated by Ca2+-Regulated Exocytosis of Lysosomes. Cell 106, 157-169.
45. Mattson, M. P. (2004). Pathways towards and away from Alzheimer's disease. 430, 631-639.
46. Kawahara, M., Kuroda, Y., Arispe, N. & Rojas, E. (2000). Alzheimer's beta-Amyloid, Human Islet Amylin, and Prion Protein Fragment Evoke Intracellular Free Calcium Elevations by a Common Mechanism in a Hypothalamic GnRH Neuronal Cell Line. J. Biol. Chem. 275, 14077-14083.
47. LaFerla, F. M. (2002). Calcium dyshomeostasis and intracellular signalling in Alzheimer's disease. Nat Rev Neurosci 3, 862-72.
48. Rapoport, N., Marin, A. P. & Timoshin, A. A. (2000). Effect of a polymeric surfactant on electron transport in HL-60 cells. Arch Biochem Biophys 384, 100-8.
49. Marks, J. D., Pan, C. Y., Bushell, T., Cromie, W. & Lee, R. C. (2001). Amphiphilic, tri-block copolymers provide potent membrane-targeted neuroprotection. Faseb J 15, 1107-9.
50. Singh-Joy, S. D. & McLain, V. C. (2008). Safety assessment of poloxamers 101, 105, 108, 122, 123, 124, 181, 182, 183, 184, 185, 188, 212, 215, 217, 231, 234, 235, 237, 238, 282, 284, 288, 331, 333, 334, 335, 338, 401, 402, 403, and 407, poloxamer 105 benzoate, and poloxamer 182 dibenzoate as used in cosmetics. Int J Toxicol 27 Suppl 2, 93-128.

1. A method of treating amyloid oligomer toxicity comprising contacting a cell comprising an amyloid oligomer with an effective amount of a poloxamer.

2. The method of claim 1, wherein the poloxamer has the general structure: CH₃(OCH₂CH₂)ₐ(OCHCH₃CH₂)_{b}(OCH₂CH₂)ₐOH, wherein a is 60 to 90, and b is 20, 21, 22, 23, 24, 25, 26, 27, 28, 29, 30, 31, 32, 33, 34, 35, 36, 37, 38, 39, or 40.

3. The method of claim 1, wherein the poloxamer is poloxamer P188.

4. The method of claim 1, wherein the amyloid oligomer is an oligomer of amyloid β, islet amyloid polypeptide (IAPP), synuclein, or prion oligomer.

5. The method of claim 4, wherein the amyloid oligomer is an amyloid oligomer.

6. The method of claim 1, wherein the cell is in a subject.

7. A method of treating a subject having Alzheimer's disease comprising administering to a subject having Alzheimer's disease with an effective amount of a poloxamer.

8. The method of claim 7, wherein the poloxamer has the general structure: CH₃(OCH₂CH₂)ₐ(OCHCH₃CH₂)_{b}(OCH₂CH₂)ₐOH, wherein a is 60 to 90, and b is 20, 21, 22, 23, 24, 25, 26, 27, 28, 29, 30, 31, 32, 33, 34, 35, 36, 37, 38, 39, or 40.

9. The method of claim 7, wherein the poloxamer is poloxamer P188.

10. A method of treating a protein mis-folding disorder comprising administering to a subject having protein mis-folding disorder an effective amount of a poloxamer.

11. The method of claim 10, wherein the poloxamer has the general structure: CH₃(OCH₂CH₂)ₐ(OCHCH₃CH₂)_{b}(OCH₂CH₂)ₐOH, wherein a is 60 to 90, and b is 20, 21, 22, 23, 24, 25, 26, 27, 28, 29, 30, 31, 32, 33, 34, 35, 36, 37, 38, 39, or 40.

12. The method of claim 10, wherein the poloxamer is poloxamer P188.

13. The method of claim 1, wherein the protein mis-folding disorder is Alzheimer's disease, type II diabetes, or Parkinson's disease.

14. The method of claim 4, wherein the protein mis-folding disorder is Alzheimer's disease.
